(19) United States
(12) Patent Application Publication
(10) Pub. No.: US 2017/0106020 A1
(43) Pub. Date: Apr. 20, 2017

(54) ANESTHETIC/ANALGESIC COMPOSITION

(71) Applicant: Stanley James Harless, Papillion, NE (US)

(72) Inventor: Stanley James Harless, Papillion, NE (US)

(21) Appl. No.: 15/295,898

(22) Filed: Oct. 17, 2016

Related U.S. Application Data
(60) Provisional application No. 62/243,539, filed on Oct. 19, 2015.

(51) Int. Cl. A61K 33/38; A61K 47/14; A61K 47/44; A61K 47/12; A61K 31/045; A61K 45/06; A61K 47/26; A61K 47/18; A61K 47/24; A61K 47/10

(52) U.S. Cl. CPC A61K 33/38; A61K 47/24; A61K 47/14; A61K 47/44; A61K 47/10; A61K 31/045; A61K 45/06; A61K 47/26; A61K 47/183; A61K 47/12

(57) ABSTRACT

Some embodiments of the present disclosure include an anesthetic/analgesic composition for preventing or treating infections of the skin or muscle, insect bites, bruises, contusions, lacerations, burns, and other inflammatory conditions, including arthritic pain of joints. The composition may include water; a member selected from the group consisting of ionic silver and nano-silver; a phospholipid; a di-glyceride; a mineral oil; a humectant; a curcuminoid; and optionally, menthol.

### RELATED APPLICATION

This application claims priority to provisional patent application U.S. Ser. No. 62/243,539 filed on Oct. 19, 2015, the entire contents of which is herein incorporated by reference.

### BACKGROUND

The embodiments herein relate generally to anesthetic/analgesic compositions, and more particularly, to anon-toxic, natural topical cream or spray for cuts, contusions, bruises, inflammatory skin reactions, such as insects' bites and allergic reactions, and the relief of arthritic joint pain, wherein the cream or spray may also be bactericidal, viricidal, and fungicidal.

Cuts and contusions of the skin and muscle may result in bacterial infections, which delays the healing time. Antibiotics take advantage of the difference between the structure of the bacterial outer cell membrane and the host's outer cell membrane. Antibiotics prevent bacterial cells from multiplying so that the bacterial population remains the same, allowing time for the host's defense mechanism to fight the infection or kill the bacteria, or by inhibiting the pathway needed for rebuilding their cell membranes and membranes of the organelles within the cell.

Bacteria are termed drug-resistance when they are no longer inhibited by an antibiotic to which they were previously sensitive. The emergence and spread of antibiotic-resistance bacteria has continued to grow due to both the overuse and misuse of antibiotics. This problem is not reversible.

Antibiotics also contaminate the environment, water, and sewage systems over time, providing constant exposure between the antibiotic residues and bacteria, stimulating the bacteria to build resistance.

Therefore, what is needed is a non-toxic and natural topical anesthetic/analgesic class of compound that can prevent infection or treat existing infections, while simultaneously preventing bacteria from developing an immune response to the various components of the compound and reducing pain and inflammation when applied topically.

### SUMMARY

Some embodiments of the present disclosure include an anesthetic/analgesic composition for preventing or treating infections of the skin or muscle, insect bites, bruises, contusions, lacerations, burns, and other inflammatory conditions, including arthritic pain of joints. The composition may include water; a member selected from the group consisting of ionic silver; a phospholipid; a di-glyceride; a mineral oil; a humectant; a curcuminoid; and optionally, menthol.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The cream or spray of the present disclosure may be used to prevent or treat infections of the skin or muscle, diminish arthritic pain in joints, and alleviate the pain from cuts, abrasions, bruises, insect bites, burns, and the like and may comprise the following elements. This list of possible constituent elements is intended to be exemplary only, and it is not intended that this list be used to limit the cream or spray of the present application to just these elements. Persons having ordinary skill in the art relevant to the present disclosure may understand there to be equivalent elements that may be substituted within the present disclosure without changing the essential function or efficacy of the composition.
1. Water
2. Ionic Silver+ (Ag⁺)
3. Phospholipids
4. Non-polar Mineral Oil
5. Glycerin
6. Di-Glycerides
7. Curcuminoids, including purified oil of curcuminoids
8. Menthol

The various elements of the compound of the present disclosure may be related in the following exemplary fashion. It is not intended to limit the scope or nature of the relationships between the various elements and the following examples are presented as illustrative examples only.

By way of example, some embodiments of the present disclosure include an anesthetic/analgesic composition for preventing or treating infections of the skin, the antiseptic composition comprising a mixture of ionic silver+, phospholipids, such as phospholipids derived from soy beans which may be bio-identical to the phospholipids found in the mammalian cell membrane, di-glycerides, mineral oil, glycerin, and purified curcuminoids. In embodiments, the composition may also comprise menthol. Some embodiments further comprise a bactericidal and/or a fungicidal element. The composition may vary in its consistency and, thus, the anesthetic/analgesic composition may be in the form of, for example, a cream, an ointment, a spray, or the like.

In some embodiments, the anesthetic/analgesic composition may comprise about 30 to about 50 wt. % ionic silver+ solution; about 20 to about 30 wt. % humectant, such as glycerin; about 0.050 to about 0.25 wt. % methyl parabens; about 0.10 to about 2.0 wt. % lecithin (de-oiled), wherein the lecithin may be of plant origin; about 0.05 to about 1.0 wt. % polysorbate 80; about 0.05 to about 1.0 wt. % polysorbate 20; about 0.002 to about 1.0 wt. % cucurminoids; about 0.20 to about 4.0 wt. % menthol crystals, which are an antibacterial agent against several types of Streptococci and Lactobacilli and which may be synthetic or naturally formed; an optional stabilizer, such as arginine (either a salt or a base), which may allow for the reduction of the pH without redox reaction to the ionic silver solution; an optional buffer product, such as calcium salts, potassium salts, or magnesium salts, or solution salts, within a required or desired PkA; and about 0.01 to about 1.0 wt. % of an optional inhibitor, such as zinc citrate.

In embodiments, the ionic silver+ solution may be formed in a solution of water by passing a small amount, such than less than 20 milliamps, of electric current between two 99.99% pure silver electrodes immersed in 1000 mL of water, resulting in an ionic silver solution comprising a maximum of about 20 parts per million (ppm) ionic silver in 1 million milligrams (mg) of water.

Examples of suitable curcuminoids for use in the composition include turmeric extracted from the dried rhizome of Curcuma longa, which may include various curcuminoids, of which curcumin may be most important in terms of the present disclosure.

Ionic silver+ may be effective as a preservative and antiseptic, because ions are the smallest form of silver, and the efficacy of silver is dependent upon surface area in contact with microbes. The greater the surface area, the more effective the silver may be in destroying the organism. Thus, because the highest aggregate surface area of silver is ionic silver, the ionic silver+ may be very effective when used topically.

In some embodiments, the composition may comprise about 10 ppm of ionic silver+. The phospholipids may form liposomes, which may also be referred to as micelles, wherein the bio-identical phospholipids may enhance the repair of the injured lipid bilayer of the cell membranes, and simultaneously preventing the ionic silver+ from combining with negative charged molecules, like chloride. The use of the phospholipids found in lecithin to form micelles may be considered a unique biological compartmentalization transport system, allowing the ionic silver+ to be effective in the destruction of the microbes by being attracted to the negatively charged surface of the microbe cell membrane without gradually producing silver resistance. This “Kill on Contact” may be unique to ionic silver+ and may prevent silver resistance to develop in bacteria.

The di-glycerides may be added for their ability to add moisture to the skin tissue, making the skin tissue softer for quicker healing. The mineral oil may aid the composition in sticking to the skin tissue. Glycerin is a humectant, which may keep the skin moistened, that also has strong anti-inflammatory abilities. The purified oil of curcuminoids, which may be derived from turmeric, may also have anti-inflammatory and pain-reducing properties. A number of studies have been conducted that support curcumin-mediated regulation of cyclooxygenase (COX) and lipoxygenase (LOX) pathways, which may be the result of the natural or inherent ability of curcumin to block the production of CO/LOX without adverse side effects involving ulcers forming in the digestive system, as compared to NSAIDs. See Maroon, Joseph et al., Natural anti-inflammatory agents for pain relief, Surg. Neurol. Int. 2010; 1:80. This is an important advantage over synthetic COX/LOX inhibitors, such as non-steroidal anti-inflammatory drugs. See Leone S. et al., Dual acting anti-inflammatory drugs, Curr Top Med Chem, 2007; 7(3): 265-75.

As stated above, ionic silver+ by definition means silver ions carrying a positive charge, and may be bactericidal, virucidal, and fungicidal. Bacteria, fungus, molds, and viruses, including Methicillin Resistant Staph Aureus (MRSA), Pseudomonas, aeruginosa, and E. coli, are sensitive to ionic silver. Canada albicans, Aspergillus brasiliensis, and viruses may also be controlled with ionic silver+.

To manufacture the composition of the present disclosure, the ingredients may be slowly mixed at room temperature to prevent breaking of the liposomes, resulting in a fixed system or set emulsion. For example, a mixing vessel may be charged with an ionic silver+ solution and zinc oxide may be added to the solution until it is dissolved. Glycerin methyl parabens and menthol crystals may be premixed with heat between about 90° and about 140° until a solution is formed. Curcumin material, such as curcuminoids extracted from turmeric includes curcumin from the dried rhizome of Curcuma longa, may be added to polysorbate and mixed until a substantially clear solution is created.

The main mixer may be started as a sheer-type mixer and the glycerin premix may be added to the main vessel of ionic silver. A homogenizer may be started, running at about 7500 to about 30,000 rpm, allowing the material to pass through before adding lecithin thereto. The lecithin may be slowly titrated within the system, allowing micelles to form. The process may continue until the micelle is between 3-10 nm in size. The pH of the solution may be stabilized to a value between 4.5 and 8.5 by adding mineral salt to the solution. The pre-blended curcumin may be added into the mixer, while using sheer mixing, allowing uniform dispersion around the outer layer of the micelle. Arginine salt or acid may be added to the mixer to establish a pH level of from about 6.5 to about 8.0. In the case of a loose emulsion, polysorbate 20 may be added at low levels for high lipid balance (HLB) ration from addition of lecithin.

Using the composition of the present disclosure may comprise gently cleaning the area to be treated, for example with saline and gauze, and applying the composition to the skin sparingly, leaving the area open to air or covering the area with a bandage. The application of the composition may increase the control of infection and promoting healing in cuts, abrasions, contusions, bed sores, pressure sores, surgical incisions, granulating wounds, hot spots and lick granulomas in dogs, and the like.

The composition may be sparingly applied to a general area of skin to relieve pain from arthritis, muscle soreness, sprains, and superficial infections. It may be beneficial to cleanse the areas with a mild soap and water before applying the composition and massage the composition into the skin after it is applied until the surface of the skin is only slightly damp. After several minutes, any excess product may be removed with a clean cloth, and clothing may be put on over the area. For severe inflammation, it may be beneficial to apply the composition multiple times, such as 2-3 times, per day. For chronic conditions, it may be beneficial to apply the composition once daily.

### Example

A test composition comprising 50% silver ionic solution, 44.5% glycerin, 0.150% methyl parabens, 2.0% de-oiled lecithin, 1.0% polysorbate 80; 0.5% polysorbate 20, 0.50% curcuminoids; and 1.25% menthol crystals was prepared in spray form. The spray underwent a United States Pharmacopeial (USP) <51> test, which quantitatively assess antimicrobial preservatives added to non-sterile dosage forms.

Five test microorganism(s), Staphylococcus aureus 6538, Aspergillus brasiliensis 16404, Pseudomonas aeruginosa 9027, Candida albicans 10231, Escherichia coli 8739, were selected for the test.

General Procedure: The test microorganisms were prepared by growth in liquid or agar culture medium. Microorganisms grown in liquid culture were centrifuged and washed prior to the test. Suspensions of the test microorganisms were standardized by dilution in a buffered saline solution. The test and control substances were dispensed, in similar known volumes, to sterile vessels. Independent volumes of the Test and Control substances were inoculated with each test microorganism mixed and incubated. Control substances were immediately harvested and represent the concentration present at the start of the test, or time zero. Incubated Test Substances were harvested at the conclusion of each contact time by chemical neutralization. The number of surviving microorganisms at the respective contact times were assessed and logarithmic reductions were calculated based on initial concentrations observed at time zero.

Passing Criteria: Criteria for antimicrobial effectiveness is determined based on the category to which a substance belongs. For Category 2 products, the criteria for bacteria is not less than 2-log₁₀ from the initial count at 14 days, and no increase from the 14 day count at 28 days. The criteria for yeast and mold is no increase from the initial count at 14 and 28 days.

Specific Testing Parameters Used:
Test Substance Volume: 10 mL
Replicates: Single
Control Substance: PBS (10 mL)
Culture Growth Media: Tryptic Soy Broth (bacteria) and Potato Dextrose Agar (Yeast and Fungi)
Culture Growth Time: 18-24 hours (bacteria), 48 hours (yeast), 7 days (fungi)
Plating media: Tryptic soy agar (bacteria) and potato dextrose agar (yeast and fungi)
Inoculum Concentration: 1.0×10⁵ CFU/mL
Inoculum Volume: 0.05 mL
Observation Times: 14 and 28 days
Volume harvested: 0.100 mL
Enumeration plate incubation temperature: 36°±1° C. (bacteria), 30°±1° C. (yeast and fungi)

Control results: neutralization method was verified. The media was sterile, and growth was confirmed (morphology on growth media).

Calculations:

$${log}_{10} Reduction = log \frac{B}{A}$$

Where B is the number of viable test microorganisms in the control substances immediately after inoculation and A is the number of viable test microorganisms in the test substances after the contact time.

Results:

| | Test Microorganism | | | | | | |
|---|---|---|---|---|---|---|---|
| Test | Contact | Data | E. coli | P. aeruginosa | S. aureus | A. brasiliensis | C. albicans |
| Substance | Time | Description | 8739 | 9027 | 6538 | 16404 | 10231 |
| Spray | Time | CFU/mL | 4.8 × 10⁵ | 4.8 × 10⁵ | 8.9 × 10⁵ | 3.85 × 10⁵ | 5.9 × 10⁵ |
| | Zero | | | | | | |
| | Day 14 | CFU/mL | <5 × 10¹ | <5 × 10¹ | <5 × 10¹ | 1.0 × 10² | <5 × 10¹ |
| | | Log₁₀ | >3.98 | >3.93 | >4.25 | 3.59 | >4.07 |
| | | Reduction | | | | | |
| | Day 28 | CFU/mL | <5 × 10¹ | <5 × 10¹ | <5 × 10¹ | <5 × 10¹ | <5 × 10¹ |
| | | Log₁₀ | >3.98 | >3.93 | >4.25 | >3.89 | >4.07 |
| | | Reduction | | | | | |

CFU = colony forming unit

Conclusion: The test sample was tested per USP <51>and successfully met the passing criteria. The test sample was able to reduce the bacteria and yeast down to non-detectable levels after 14 days. The mold was reduced to non-detectable levels after 28 days.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A composition for preventing or treating infections of the skin, the composition comprising:
water;
a member selected from the group consisting of ionic silver and nano-silver;
a phospholipid;
a di-glyceride;
a mineral oil;
a humectant;
a curcuminoid; and
optionally, menthol.

2. The composition of claim 1, wherein the phospholipids are derived from soy beans.

3. The composition of claim 1, further comprising a bactericidal.

4. The composition of claim 1, further comprising a fungicidal.

5. The composition of claim 1, further comprising polysorbate.

6. The composition of claim 1, comprising:
about 30 to about 50 wt. % silver ionic solution;
about 20 to about 35 wt. % glycerin;
about 0.050 to about 0.25 wt. % methyl parabens;
about 0.10 to about 2.0 wt. % lecithin (de-oiled);
about 0.05 to about 1.0 wt. % polysorbate 80;
about 0.05 to about 1.0 wt. % polysorbate 20;
about 0.002 to about 1.0 wt. % cucurminoids;
about 0.20 to about 4.0 wt. % menthol crystals;
an optional stabilizer;
an optional buffer product; and
about 0.01 to about 1.0 wt. % of an optional inhibitor.

7. The composition of claim 6, wherein the optional stabilizer comprises arginine.

8. The composition of claim 6, wherein the optional buffer product comprises a member selected from the group consisting of calcium salts, potassium salts, and magnesium salts.

9. The composition of claim 6, wherein the optional inhibitor comprises zinc citrate.
